(19) United States
(12) Patent Application Publication
(10) Pub. No.: US 2004/0133450 A1
(43) Pub. Date: Jul. 8, 2004

(54) Control system and method intended to be used when performing clinical trials

(76) Inventors: Ola Foureaux, Kungsbacka (SE); Marcus Kappi, Goteborg (SE)

Correspondence Address: MATHEWS, COLLINS, SHEPHERD & MCKAY, P.A., 100 THANET CIRCLE, SUITE 306, PRINCETON, NJ, 08540-3674, US

(21) Appl. No.: 10/468,137

(22) PCT Filed: Feb. 19, 2002
(86) PCT No.: PCT/SE02/00297

(30) Foreign Application Priority Data
Feb. 19, 2001 (SE) 010060603

(51) Int. Cl. G06F 17/60

(52) U.S. Cl. 705/2; 705/5

(57) ABSTRACT

The present invention relates to a control system (10) intended for use in the performance of clinical trials. The control system (10) comprises at leat one data storage unit (12) for storing patient information and recorded or calculated data. The control system (10) also comprises a data acquisition means (14), connected to at least one of the said data storage units (12), that at least collects data excluding or including patients. The control system (10) further comprises a control means (16), connected to at least one of the said data storage units (12), that, on the basis of collected data, books patient appointments or excludes patients through the application of at least one predefined boundary condition and/or a predefined target function, M(p), p designating different parameters. The control system (10) further comprises an appointment-booking means (18), connected to the control means (16). It is then no longer necessary to recruit any new patients for a clinical trial when the control means (16) determines that the target function M(p) has been met or that an error function E=f(M, U) is less than a predetermined threshold value in which U(p) designates the real outcome.

### FIELD OF INVENTION

The present invention relates to a first aspect of a control system intended for use in performing clinical trials.

According to a second aspect, the present invention relates to a method intended for use in performing clinical trials.

According to a third aspect, the invention relates to at least one computer program product intended for use in performing clinical trials.

### BACKGROUND OF THE INVENTION

Clinical trials are studies whose objective is to achieve scientific elucidation of the efficacy of treatments, most often with pharmaceutical drugs. These studies are traditionally subdivided into different phases, i.e. Phase I, Phase II, Phase III and Phase IV. Phase III is particularly interesting in this context. A Phase III trial is a large study whose aim is to achieve the licensing of a new pharmaceutical drug on the market. A phase III trial must often employ numerous patients for statistical proof of the drug's efficacy.

These studies with numerous patients are performed by necessity at a plurality of centres (clinics) and often in a plurality of countries as well. These multicentre studies are traditionally conducted by having each centre recruit its own patients. Once the patients have commenced treatment, all the patients must undergo the same treatment for an equally long period of time. This means that the sooner the last patient has commenced treatment, the sooner the study will be completed.

Before any patient enters the treatment phase, she/he must pass through the recruitment phase and the screening phase. FIG. 1 illustrates the different steps leading to the start of treatment:

F₁ designates the recruitment phase in which a ‘pool’ of interested patients Pro is collected. Possible patients, Pᵣ₁, are then selected from this ‘pool’. In the next phase, i.e. the screening phase, Fₛ, a number of patients (Nₛ₀) are summoned to an examination (Uₛ) whose purpose is to select suitable patients. Patients not excluded (Nₛ₁) move on and form the pool (Pₛ). N_{b0} patients from this pool (Pₛ) are summoned to the start of treatment and the treatment phase. Those not excluded (N_{b1}) form the pool of treatment patients (P_{b}).

Each such phase may entail a number of different examinations. Each centre (clinic) has traditionally administered and planned these phases locally.

As a result, the recruitment phase is often protracted, and recruitment may take several years.

A sufficient number of patients must be treated if a study is to prove a drug's efficacy. For participation in a study, a patient must meet a number of inclusion criteria and fail to meet a number of exclusion criteria. For example, an inclusion criterion may stipulate that the patient must lie within a specific age range. Drug addition or hypertension are examples of exclusion criteria.

The number of patients needed for screening examinations in order to obtain enough patient for starting treatment is also often misjudged when too many patients meet the exclusion criteria, fail to meet the inclusion criteria or fail to show up for examination. This leads to additional recruitment and a prolonged duration for the study's implementation. This is because the study is not completed until the last treated patient has completed the entire treatment, regimen stipulated in the clinical trial protocol.

The clinical trial protocol is a detailed description of the clinical trial at the application stage.

In addition, targets are sometimes specified for the magnitude of different patient categories for each centre, even though these targets actually apply to the study as a whole. This also makes it harder to recruit a sufficient number of patients quickly. For example, the targets of 50% men and 50% women may be set for each centre, even though they apply to the study as a whole. Another example might be to have 10% of the group consist of diabetics, a target for the study as a whole.

### SUMMARY OF THE INVENTION

One purpose of the present invention is to solve the aforementioned problems.

In accordance with the present invention, a first aspect is achieved of a control system intended for use in performing clinical trials. The control system comprises at least one data storage unit for storing patient information and recorded or calculated data. The control system also comprises at least one data acquisition means, connected to at least one of the said data storage units that at least collects data excluding and/or including patients. The control system also comprises a control means, connected to at least one of the said data storage units, which, on the basis of collected data, books patient appointments or excludes patients by the use a predefined boundary condition and/or a predefined target function, M(p). The control system also comprises an appointment-booking means, connected to the control means, for booking patient appointments. No new patients need to be recruited for a clinical trial when the control means determines that the target function M(p) has been met or that an error function E=f(M, U) is less than a predefined threshold value in which U(p) designates the actual outcome. This control system greatly improves the speed at which patients enter the study. This means that the study's implementation duration is clearly shortened, saving a great deal of money. In addition, the degree of target achievement for different patient categories is improved and offers patients opportunities for earlier treatment.

In this context, it would be advantageous if the error function were:

$$E = {\sum }_{m = 1}^{{m}_{max}} {[ \vert U ( {p}_{m} ) - M ( {p}_{m} ) \vert * w ( {p}_{m} ) ]}^{n}$$

in which U(pₘ) is the actual outcome, n=1, and w(pₘ) is a weighting function designating the degree of importance of meeting the parameter pₘ's target value.

An additional advantage is achieved in this context if the initial part of each clinical trial were performed in three consecutive phases. In the first phase, the control means selects a subset, Pᵣ₁, of possible and/or desirable patients from a set Pᵣ₀ of interested participants stored in the data storage unit. In the a second phase, a number of patients, Nₛ₀ from the set, Pᵣ₁, of possible patients is summoned, in one or more steps, to an examination, whereupon the control means excludes unsuitable patients on the basis of examination results, resulting in a set, Pₛ, of patients. In the third phase, a number of patients, N_{b0}, from the set, Pₛ, are summoned to the start of treatment and examination, whereupon the control means excludes unsuitable patients on the basis of the examination results, resulting in a set, P_{b}, of patients to receive treatment.

In this context, it would be advantageous if the initially summoned patients, Nₛ₀, were determined by the following relationship:

Nₛ₀=P₁/(K₁*K₂*K₃* . . . *Kₙ)

in which P₁ designates the number of patients who need to begin treatment in the said clinical trial, and Kᵢₓ, in which 1=i=n is an integer and 1=x=m, m being an integer in which x designates different patient categories, designates an estimated system constant indicating the percentage of patients who were not excluded after each step, i, and in which 0=Kᵢₓ=1.

An additional advantage is achieved in this context if the control means corrects the system constants, Kᵢₓ, after a sufficient number of patients has completed one or more of the steps set forth in claim 4.

In this context, it would be an advantage if the control means adjusted the number and composition of patients summoned to examinations until the clinical trial's treatment start is deemed to have been concluded.

An additional advantage is achieved in this context if the said parameters could e.g. be the number of patients commencing treatment, gender distribution, age distribution and he composition of patients based on other patient values.

It would be advantageous in this context if the said boundary condition could be e.g. that the age of the patient must lie within an open or closed predetermined range, that different patient values, such as laboratory results, lie within predetermined open or closed intervals and that certain diagnoses are met or not met.

An additional advantage is achieved in this context if at least one of the data storage units consisted of a database.

Another objective of the present invention is to achieve a method intended for use in performing clinical trials. The method comprises the following steps:

Defining a target function, M(p), encompassing a number of target values and in which p designates different parameters;

Estimating system constants, Kᵢₓ, in which 1=i=n and n is an integer, and 1=x=m and m is an integer, x designating different patient categories indicating the percentage of patients who were not excluded after each stage, i, of the clinical trial and in which 0=Kᵢₓ=1;

Defining a data set for collection in each examination;

Defining a boundary condition, rᵢⱼ, in which i designates the examination no., i, 1=i=n, n being an integer, and j designates different boundary conditions for examination, i, 1=j=m, m being an integer;

Selecting, from at least one data storage unit storing patient data, a set, Pᵣ₁, of possible patients, whose number is selected according to the number of patients who need to undergo treatment in the said clinical trial, and at least one exclusion criterion, r₀ⱼ;

Selecting, with the aid of boundary conditions, rᵢⱼ, possible patients for the next examination, i, no. i+1;

Including, on the basis of examination results, suitable patients, resulting in a set, Pₛ, of patients;

Correcting the system constants, Kᵢₓ, on the basis of the actual outcome U(p);

Selecting patients from the set, Pₛ, to summon to the start of treatment, N_{b0};

Applying the boundary condition, rᵢⱼ, and excluding or including patients on the basis of the examination at the start of treatment, resulting in a set, P_{b}, of patients

Possibly correcting the system coordinates, Kᵢₓ, on the basis of the actual outcome U(p);

Repeating the six preceding steps until the target function M(p) has been met or an error function E=f(M, U) is less than a predetermined threshold value in which U(p) designates the actual outcome.

The speed with which all patients enter the study will be greatly improved with this method. As a result, the study's implementation duration will be clearly shortened, saving considerable expense. Moreover the degree of target achievement for different categories of patients will be improved and offer patients opportunities for earlier treatment.

In this context, it would be advantageous if the error function were

$$E = {\sum }_{m = 1}^{{m}_{max}} {[ \vert U ( {p}_{m} ) - M ( {p}_{m} ) \vert * w ( {p}_{m} ) ]}^{n}$$

in which U(pₘ) is the actual outcome, n=1, and w(pₘ) is a weighting function designating the degree of importance of meeting the parameter pₘ's target value.

An additional advantage is achieved in this context if an initial number of patients, Nₛ₀, is established from the following correlations:

Nₛ₀=P₁/(K₁*K₂*K₃* . . . *Kₙ)

In this context, the use of an error function E, =f(M,U), would be useful in evaluating the priority of the parameters, pₘ, when several parameters in the target function, M(pₘ) are unable to reach their targets at the same time.

An additional advantage is achieved in this context when the clinical trial is devised as a multicentre study.

Another objective of the present invention is to achieve at least one computer program product that can be downloaded into the internal memory of at least one digital computer. At least one of the said computer program products comprises software code for performing the steps in the method according to the present invention when at least one product is run on at least one of the said computers.

The speed at which all the patients enter the study will be greatly increased with at least one of the computer program products. As a result, the study's implementation duration will be clearly shortened, saving considerable expense. This will also increase the possibility of meeting demands for statistical significance. Moreover the degree of target achievement for different categories of patients will be improved and offer patients opportunities for earlier treatment.

Embodiments of the invention will now be described, referring to the attached drawings in which:

### BRIEF DESCRIPTION OF THE DRAWINGS

FIG. 1 is a schematic view of the different steps leading to the start of treatment in a clinical trial according to the prior art;

FIG. 2 is a block diagram of a control system intended for use in performing clinical trials according to the present invention;

FIG. 3 is a block diagram of the control system shown in FIG. 2 introduced into the steps shown in FIG. 1;

FIG. 4 is a flow chart of a method, according to the present invention, intended for use in performing clinical trials; and

FIG. 5 is a schematic view of some computer program products according to the present invention.

### DETAILED DESCRIPTION OF EMBODIMENTS

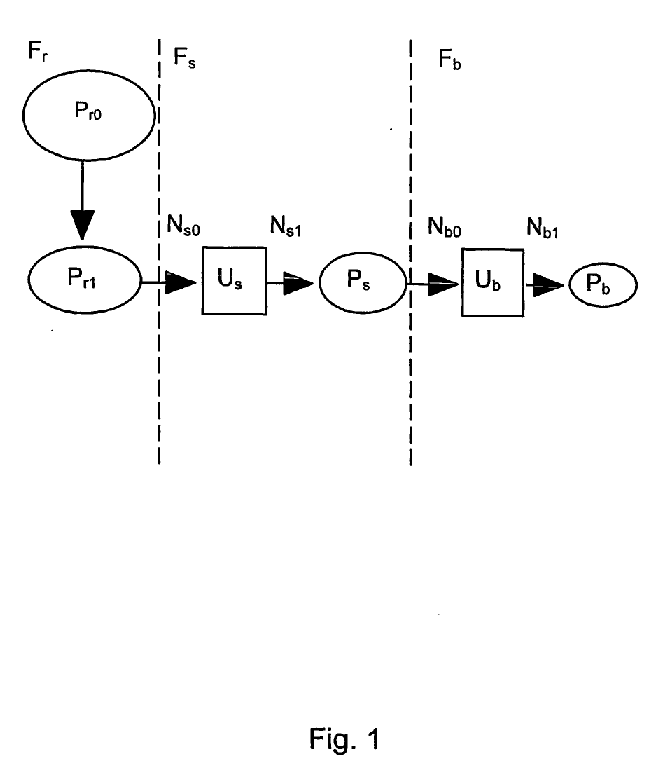

FIG. 1, which schematically depicts the different steps preceding the start of treatment in a clinical trial according to the prior art, was previously described under the heading “Background of the invention” and will not be further described here.

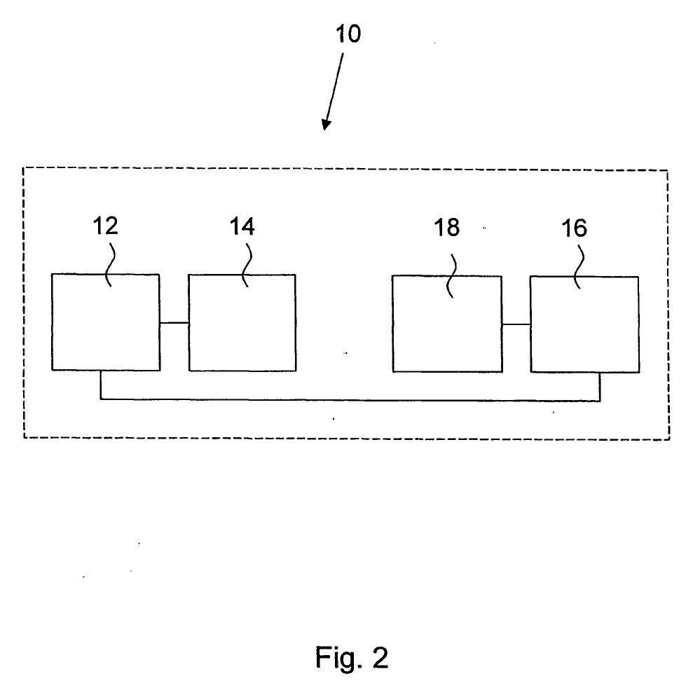

FIG. 2 is a block diagram of a control system 10 according to the present invention. The control system 10 is intended for use in performing clinical trials. The control system 10 comprises at least one data storage unit 12 for storing recorded or calculated patient information. For the sake of simplicity, only one data storage unit 12 is shown in FIG. 2. The control system 10 also comprises a data acquisition means 14, connected to the data storage unit 12, which e.g. collects data excluding or including patients. The data acquisition means 14 also collects examination data. The data storage unit 12 can e.g. consist of a database or a structured file. The control system 10 also comprises control means 16, connected to the data storage unit 12, able to book patient appointments, on the basis of collected data, or exclude patients by applying at least one predefined boundary condition and/or a predefined target function, M(p) in which p designates different parameters. One parameter p may stipulate e.g. a gender distribution across all centres of 60% of the women who start treatment. The control system 10 also comprises an appointment-booking means 18, connected to the control means 16, for booking patient appointments. No new patients need to be recruited to a clinical trial using this control system 10 when the control means 16 determines that the target function M(p) has been met or an error function E=f(M, U) is less that a predetermined threshold value, U(p) designating the actual outcome. This means that the recruitment phase and the screening phase have been concluded in the clinical trial (cf. FIG. 1). However, the treatment phase has not been concluded.

The said error function can be described with the following equation:

$$E = {\sum }_{m = 1}^{{m}_{max}} {[ \vert U ( {p}_{m} ) - M ( {p}_{m} ) \vert * w ( {p}_{m} ) ]}^{n}$$

in which U(pₘ) is the actual outcome, n=1, and w(pₘ) is a weighting function designating the degree of importance of meeting the parameter, pₘ.

An example of the said error function is obtained if n=2, i.e.

$$E = {\sum }_{m = 1}^{{m}_{max}} {[ \vert U ( {p}_{m} ) - M ( {p}_{m} ) \vert * w ( {p}_{m} ) ]}^{2}$$

The said control means 16 corrects a number of system constants, Kᵢₓ, when a predetermined number of patients have passed the said start of treatment. Kᵢₓ designates an initially estimated system constant designating the percentage of patients who, after each step, were not excluded. At the same time 0=Kᵢₓ=1 and 1=i=n, where n is an integer. Different constants, Kᵢₓ, can also be assumed and successively corrected in relation to different parameters pₘ according to the above.

The said control means 16 also adjusts the number of patients summoned to examinations until recruitment to the clinical trial is regarded as concluded.

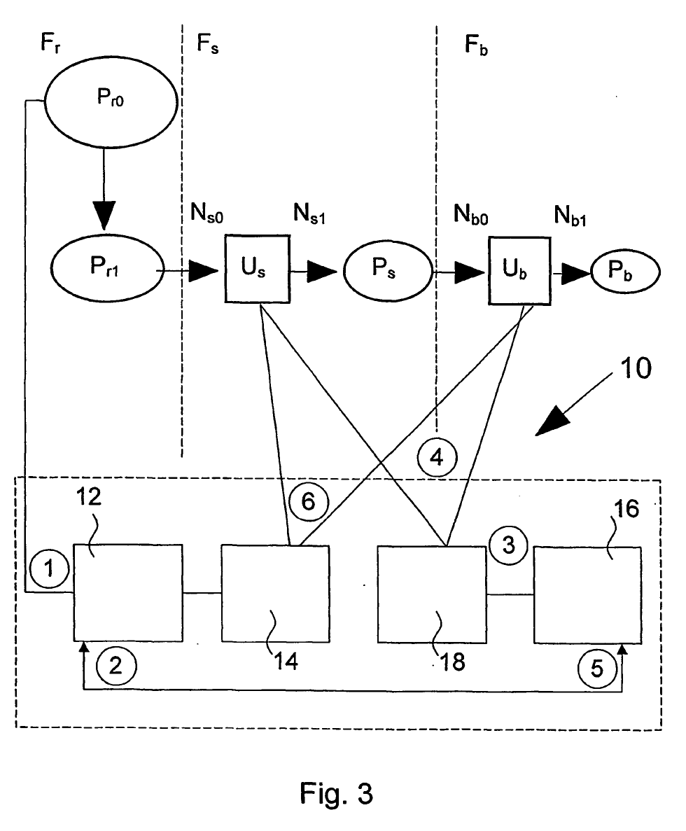

FIG. 3 is a block diagram of the control system 10, shown in FIG. 2, introduced into the steps shown in FIG. 1. FIG. 3 uses the same reference designations as in FIGS. 1 and 2.

1. This indicates that the data storage unit 12 contains information on the initial pool (Pᵣ₀). This information may have been collected via e.g. advertisements and application forms available on the Internet. The information may also have been collected by telephone, i.e. in telephone interviews. When the control means 16 has evaluated the pool, a selection of these patients is stored in a pool (Pᵣ₁) of accessible, recruitable patients. This is accomplished by an exchange of information between 2 and 5 according to the figure. Evaluation is primarily performed by applying medical exclusion criteria (limit criteria) to Pᵣ₀. Information on the patients' conditions is not always available to make this possible. Nor can all medical exclusion criteria (limit criteria) be used, i.e. only those for which values for e.g. age, height and weight are available.

2. Data are retrieved from the data storage unit 12 for making the evaluation in the control means 16. The results of calculations are also stored in the data storage unit 12.

3. When the control means 16 has selected patients to be summoned to the next examination, the appointment-booking means 18 books these appointments for them. Patients are to be summoned e.g. to examination. There are a number of ways to do this, but they are not addressed here. Here, it is merely assumed that the appointments are made and that patients are informed about the appointments and accept them. This is performed in 4.

4. See 3 above!

5. This designates all the information retrieved from the data storage unit 12 for processing in the control means 16.

6. The data acquisition unit 14 collects data on the number of patients examined and the results of their examinations. Collecting all data is not necessary. However, the data included in the boundary conditions, i.e. exclusion criteria, inclusion data and values affecting the target function must be collected for optimal operation of the control means 16. The way in which this is accomplished is not treated here. The collected values are stored in the data storage unit 12. In FIG. 3, data are collected in two steps in the patient flow. However, it should be noted that this number may vary from one trial to another.

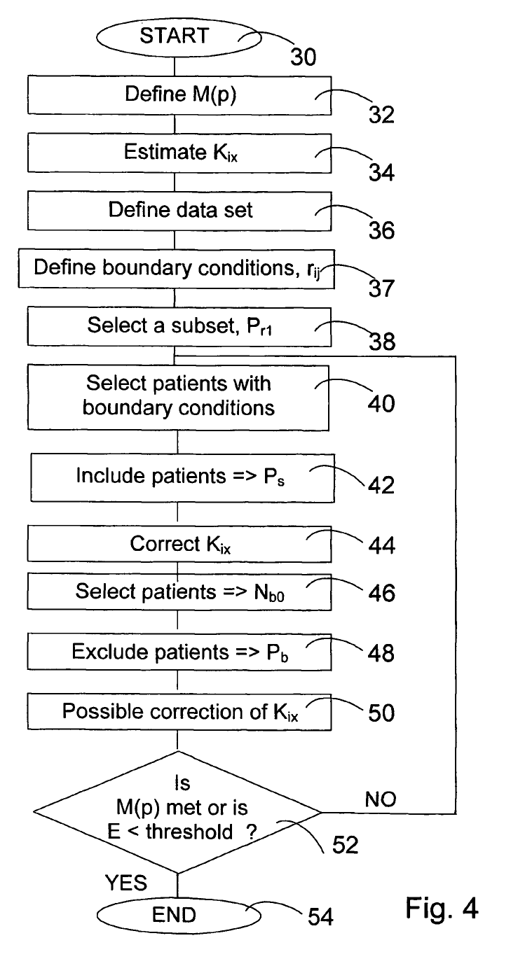

A flowchart in FIG. 4 depicts a method according to the present invention, said method being intended for use in the performance of clinical trials. The method starts at block 30. The method then continues at block 32 with a step entailing definition of a target function, M(p), comprising a number of target values p designating different parameters. The method continues thereafter at block 34 with a step entailing estimation of system constants, Kᵢₓ, in which 1=i=n, where n is an integer designating the percentage of patients who were not excluded after each step, i, in the clinical trial. Moreover, 0=Kᵢₓ=1. The method then continues at block 36 with a step entailing definition of a data set for collection in each examination. The method then continues at block 37 with a step entailing definition of a number of boundary conditions, rᵢⱼ, in which i designates the examination number, i, 1=i=n, n being an integer and j designating different boundary conditions for the examination, i, 1=j=m, m being an integer. The method then continues at block 38 entailing selection by at least one data storage unit storing patient information of a subset, Pᵣ₁, with possible and/or desirable patients the number of which is selected according to the number of patients who need to undergo treatment for the said clinical trial, and at least one exclusion criterion (boundary condition). In block 40, a step is performed in which potential patients for examination are selected with the aid of boundary conditions rᵢⱼ. The method then continues at block 42, at which suitable patients are included, resulting in a set, Pₛ, of patients. Block 44 performs a step in which the system constants, Kᵢₓ, are corrected on the basis of the actual outcome U(p). The method then continues at block 46 with a step in which patients to be summoned to the start of treatment, N_{b1}, are selected from the set, Pₛ. Block 48 performs a step in which the boundary conditions rᵢⱼ are applied, and patients are excluded or included, depending on the findings from the examination at the start of treatment, resulting in a set, P_{b}, of patients to undergo treatment. The method then continues at block 50 with a step entailing possible correction of the system constants, Kᵢₓ, on the basis of the actual outcome U(p). Block 52 asks the questions: has the target function, M(p) been met or does the error function E=f(M, U) lie below a predetermined threshold value in which U(p) designates the actual outcome? If the answer to this question is affirmative, the method concludes at block 54. However, if the answer is negative, the steps according to blocks 40-52 are repeated.

The method described in FIG. 4 can be performed e.g. with the aid of the control system shown in FIG. 2.

A different description of the initiating part of the method according to the present invention is provided below.

Description of the Method with Details and Examples

STEP 32. Defining M(p), E(U,M)

M(p)—Target Function

Define target values and limit values for the start of treatment! These target values differ from trial to trial. All target values can jointly be referred to as the target function in which the different criteria constitute the set of definitions, and desirable values or limit values constitute the set of values.

We call the target function M(p) in which the definitions set p consists of the different parameters that must have certain values or lie within certain intervals.

For example: the percentage of women, laboratory value Y greater than 10 etc. The set of values M(p) is then e.g. 60%, 20%, 10% etc.

Here are examples of definition-value set pairs in the target function M(p):

Parameters in the target function:

Parameter 1 (p₁). The number of patents beginning treatment must be 1,900.

Parameter 2 (p₂). Gender distribution across all participating centres must be 60% women. (Local distribution is allowed to vary.)

Parameter 3 (p₃). At least 10% of patients must have limit values for glucose in the range for clinical definition as IGT (Insufficient Glucose Tolerance).

M(p) can be expressed e.g. so:

M(p₁)=1900

M(P₂)=60%

M(p₃)>=10%

Additional parameters may occur in the same way as above.

A weighting function w(p) relating the degree of importance of the different criteria constituting the definition set can also be stipulated. This would make it easier to manage different deviations and set the degree of importance for different criteria in relation to each other. Certain target values are ‘non-negotiable’. E.g. the number of patients starting treatment must amount to 1,900.

E(U,M)—Error Function

The error function designates the extent to which the actual outcome, U(p), deviates from the desired outcome as indicated by the target function M(p). The actual outcome is calculated by summoning a subset of patient to examination and collecting data from it. The error function can be calculated in this way. Results are then compared to targets and estimated system constants Kᵢₓ. (See the calculation of Kᵢₓ below!) The error function can be calculated in different ways. Here, we used the following equation:

$$E = {\sum }_{m = 1}^{{m}_{max}} {[ \vert U ( {p}_{m} ) - M ( {p}_{m} ) \vert * w ( {p}_{m} ) ]}^{2}$$

w(Pₘ) is a weighting function designating the importance of sub-targets m, i.e. the degree of importance of a particular target compared to other targets. (Also see the sample calculation below!)

STEP 34. Estimating Kᵢₓ

A quantitative estimate of the examinations' system properties is necessary for utilizing the error function. A description is provided below of the assumptions upon which they are based. A system of amplifier stages or, more accurately damping stages (K=1), connected in series, is analogous to this. Uₛ and U_{b} are assumed to be the ‘damping stages’ in FIG. 1.

In order to simplify this description, we assume that we only have one patient category, i.e. we use Kᵢ instead of Kᵢₓ.

First a review of the practical assumptions yielding

Kₛ=K₁and K_{b}=K₂.

Assume the following frequencies as examples of properties of the different examinations:

Frequency 1 (f₁). The percentage of summoned patients who never show up for screening.

Frequency 2 (f₂). The percentage of patients excluded in screening.

Frequency 3 (f₃). The percentage of summoned patients who never show up for the start of treatment.

Frequency 4 (f₄). The percentage of patients summoned to the start of treatment

These percentages of patients (originally summoned in a given period) who successively remain after the departure of others can be expressed as a dampening in the system with a value in the 0-1 range. With the same index as in the example, we obtain k₁=1−f₁, K₂=1−f₂ etc. Or, generally:

kᵢ=1−f₁ (1)

The meaning of f compared to f is that the damping step, i.e. the examination can be paralleled according to different categories (men, women, age range etc.). But as a simplification, the step can be regarded as non-parallel.

A maximum value for i does not necessarily need to be four. This is because screening may encompass a plurality of different examinations. The screening phase includes at least one examination and often more than one. ‘Simpler’ examinations are commonly performed first. Generally speaking, we have the following relationship designating a first approximation of the required number (Nₛ₀) of patients initially summoned in order to meet the target value p₁:

p₁=Nₛ₀*(k₁*k₂*k₃*k₄* . . . *kₙ) (2)

Merging different reasons for the dropout of summoned patients per appointment, we obtain for a study with only one screening appointment:

K₁=k₁·k₂=the number approved from screening/the number summoned to screening (3)

K₂=k₃·k₄=the number approved from the start of treatment/the number summoned to the start of treatment (4)

Or, formally:

K₁=Nₛ₁/Nₛ₀ (5)

K₂=N_{b1}/N_{b0} (6)

p₁ in (2) is N_{b1} here. It therefore follows that

N_{b1}/Nₛ₀=K₁*K₂* . . . *Kₙ (7)

These equations express the central assumptions of practical importance in dimensioning the study. A calculation example of study dimensioning follows.

### EXAMPLE

### Dimensioning a Study and Setting Kᵢₓ

Assume there is a study in which 1,900 patients must begin treatment. (A statistical calculation of the clinical trial protocol is made in determining the number. The purpose is to recruit a sufficient number of patients to demonstrate the efficacy of a drug/treatment.) The following frequencies are set, based on knowledge of the contents of earlier studies and investigations:

Frequency 1 (f₁). The percentage of summoned patients who fail to show up for a screening: 10%.

Frequency 2 (f₂). The percentage excluded at a screening: 70%.

Frequency 3 (f₃). The percentage summoned who fail to show up for the start of treatment: 4%.

Frequency 4 (f₄). The percentage excluded at the start of treatment: 25%

According to (1) we therefore obtain:

k₁=1−f₁=0.9

k₂=1−f₂=0.3

k₃=1−f₃=0.96

k₄=1−f₄=0.75

yielding, after re-writing and using (7):

Nₛ₀=N_{b1}/(k₁*k₂*k₃*k₄)=1,900/0.9*0.3*0.96*0.75)˜9,774 patients for screening.

In order to deal with erroneous assumptions, a safety factor is often set, providing a margin for underestimation of the need. Here, a 25% margin of error was used. We can now devise a basic plan for the examinations:

The number of normal screening examinations: 9,774

The number of reserve screening appointments: 2,444 (9,774*0.25)

The number of normal start of treatment appointments: 2,639 (9,774*0.9*0.3)

The number of reserve start of treatment appointments: 660 (2,444*0.9*0.3)

These examinations are to be divided among the participating centres. Each would, ideally, carry out the same number of examinations. If this were the case, the rounded-off number of potential examinations per centre with 20 participating centres would be:

The number of normal screening examinations: 489

The number of reserve screening appointments: 123

The number of normal times for start of treatment appointments: 132

The number of reserve start of treatment appointments: 33

These examinations can be planned as a potential ‘basic schedule’. New examinations can be added or planned examinations can be cancelled as information is received on examination results. It all depends on how effectively the error functions steers the programme towards the target and how well K factors have been set in relation to the actual outcome.

At the same time as efforts are made to reach the target value for the total number of patients to begin treatment, meeting a plurality of different target values simultaneously may sometimes be desirable. They may concern e.g. a specific gender distribution, a distribution among different age intervals, a minimum of subjects with various medical values above or below certain limit values etc.

The patients can then be divided into different categories with different values for Kᵢₓ. This can then be regarded as a function, kᵢ(p), whose parameter p determines which target group is involved. It can initially be assumed that kᵢ(p) are constants, as in the example above, or vary with p based on previous experience of trials.

STEP 36. Defining the Data Set

Define the data set to be collected for each examination and from the processing of applications. This step is necessary to permit monitoring the influx of data from each patient,

For example:

At the time of application: weight, height, gender, age and address.

At screening: weight, height, gender, blood pressure and HbA_{1c}.

At the start of treatment: weight, height, blood pressure and FBG (fasting blood glucose.)

Data from applications are collected in conjunction with recruitment. These data make it possible to sort patients into different categories. Recruitment can be repeated if the number of patients is insufficient, e.g. if there are no patients in a particular area. (The address is used for matching centres and patients according to the distance to the nearest participating centre.)

STEP 37. Defining Boundary Conditions—rᵢⱼ

Boundary conditions are taken from the clinical protocol. It contains detailed descriptions of the way in which the study is to be conducted, which patients are allowed to participate (may be included in the study) and which patients who are not allowed to participate (are excluded from the study). The boundary conditions can apply generally in the study or apply to values collected from a particular examination. Assign an index to each examination, and assign an index to each boundary condition in a particular examination. This yields the sum of all boundary conditions as:

$${\sum }_{i = 0}^{n} {\sum }_{j = 1}^{k} ry$$

in which 0=i=n, where n is the number of examinations, in addition to i=0 which designates the selection step for the first examination, and 1=j=m where m is the number of boundary conditions for a particular examination, or for i−0, the conditions to be applied in the selection stage.

Here is an example of different boundary conditions:

Boundary Conditions:

Boundary condition 1 (r₀₁): Age at least 18 years.

Boundary condition 2: (rᵢ₂, iε{0,1,2}). BMI at least 26 kg/M²

Boundary condition 3: (rᵢ₃, i ε{1,2}). Patients with glucose values above the limit value—defined as IGT (Insufficient Glucose Tolerance)—are to be excluded from this study.

The practical use of boundary conditions coincides with the clinical concepts ‘inclusion criteria’ and ‘exclusion criteria’. The task of boundary conditions in this context is to protect the study protocol from violations by not including, i.e. excluding, patients who are ineligible for participation in the study.

STEP 38. Selecting a Subset, Pᵣ₁ from Pᵣ₀

Create a pool of possible patients in two steps:

1. Create an initial pool (Pᵣ₀) by selecting from replies received through www applications, ads, advertising etc. and recording certain self-reported basic data, such as age, gender, weight, height etc.

2. Create a new pool, Pᵣ₁, consisting of the appropriate and, where applicable, the most suitable respondents by applying the boundary condition r₀ⱼ to the pool Pᵣ₀. This set can now be used a recruitment source for summoning patients to the first examination.

STEPS 40, 42 and 44—Screening

Steps 40, 42 and 44 describe the procedure during the screening phase. Here, patients, selected from Pᵣ₁, are summoned to the first examination. They are examined (Uₛ), and data are collected from the examination (defined in step 36). The data sets are evaluated by applying the boundary condition, r₁ⱼ, to these data. This accordingly excludes patients (step 42), leaving a pool of patients Pₛ to be summoned to the start of treatment.

If a sufficient number of patients (i.e. a number sufficient for determining the statistical significance of the need to change values for K₁ and K₂) is examined, new values for K₁ and K₂ can be calculated (step 44).

An additional description of these steps follows.

Step 1. Select the patients for screening (Nₛ₀)

Step 2. Make appointments, collect data

Step 3. Include/Exclude patients

Step 4. Adjust the system's control parameters.

1. Screening—make a selection of patients from the initial patient pool on the basis of different criteria, make appointments for them and summon them to a screening appointment (unit 18, FIG. 3).

2. The most suitable patients are summoned to the screening to permit the start of screening examinations. ‘Most suitable’ refers to patients enabling the best target values to be achieved. A sufficient number of normal screening appointments are booked primarily. The following rules apply in determining the number of patient appointments initially required:

Rule 1 Book a sufficient number of patients so the ones still suitable for [symbol] participation in the study after the screening appointment is large enough to permit calculation of the different values for K₁(pᵢ) and K₂(pᵢ) with sufficient accuracy.

Rule 2 Make sure there is sufficient time for summoning patients to these appointments, at least a week, for example.

Patients now begin to arrive at the first screening appointment.

3. Include and exclude patients and calculate screening frequencies and the number of approved patients hitherto in relation to initially set target values. All newly registered data (unit 14, FIG. 3) are tested against the rules for exclusion and inclusion respectively (unit 16, FIG. 3). Patients excluded from participation in the study are tagged as ‘excluded’ and cannot be booked for any further appointments by the appointment-booking unit (unit 18, FIG. 3).

Calculate statistics on the number of approved/disapproved/unperformed examinations at each centre and globally. This information is used e.g. as decision-making input for step 52 below.

4. See step 52 below!

STEPS 46, 48 and 50—Start of Treatment

Steps 46, 48 and 50 describe procedure in the first part of the treatment phase, i.e. what we refer to as the start of treatment. The procedure is analogous to preceding steps 40, 42, 44. Here, patients are summoned after approved screening to the start of treatment. Treatment may last for several years with a plurality of recurrent examinations. This part is not addressed in the present application.

Patients are selected from Pₛ (step 42). They are examined (U_{b}), and data are collected from their examinations (defined in step 36). The data sets are evaluated by applying the boundary condition, r₂ⱼ, to these data. This accordingly excludes patients (step 48), yielding a pool of patients P_{b} who have begun treatment.

Examination of a sufficient number (for statistical significance) of patients makes it possible to calculate new values for K₃ and K₄ (step 50).

An additional description of these steps follows below.

Step 1. Select patients (from Pₛ) to summon to the start of treatment (U_{b})

Step 2. Make appointments and collect data. Include/Exclude patients.

Step 3. Adjust the system's control parameters.

1. Start of treatment—Select, book and summon patients included in screening to the start of treatment (unit 18, FIG. 3). Also book patients for other examinations in the study according to the established treatment plan.

The system indicates when all necessary data have been recorded for patients by using a tag in the database to create a new pool of patients available for the start of treatment. The system can now select the most suitable patients (e.g. the most healthy of the obese patients) and book them for the start of treatment.

2. All of the following examinations can be booked in conjunction with the start of treatment, as they have been stipulated in the adopted treatment plan. However, this may not always be appropriate, as in this example, as the exclusion of a large number of patients is anticipated at the start of treatment. It is therefore better to make bookings in conjunction with the treatment examination when all the necessary values are available or to have the central system do so at a later date.

3. Calculate current values for comparison with the target function. Here, we can calculate the number of patients who came to the start of treatment. Measured values can be compared and possibly generate action or proposals for actions.

Also see step 50.

Other Appointments

Book appointment and record data from the examinations. Generally speaking, other appointments are booked at the start of treatment, as the treatment plan is the same for all patients. However, bookings can be changed along the way as needed.

Step 50.—Adaptive Regulation of Examination Frequencies

Adjust the number of patients summoned to examination, in each patient category and jointly, in order to minimise deviations from established target values.

Use the information obtained in steps 44 and 50 to correct, in unit 16, the parameters adopted in Step 34.

Steps 44 and 50 are continuously modified on the basis of these results for the purpose of satisfying the target function M(p) to the greatest possible extent. Since the possibility of satisfying the target function in every respect is limited, deviations are weighted in the manner set forth below.

In an ideal world, the system constant functions, Kᵢₓ(p), for different p's are constant. This is not always the case in reality. However, this assumption may suffice as an initial measure.

Summon the first period's patients, assuming that the number, multiplied by the system constant function, meets the demands for each sub-target interpolated to the percentage of the anticipated total number of summoned patients.

Summon different categories of patients from the patient pool in such a way that each category, individually or collectively, meets the target function to the greatest extent possible. The weighting function w(p) stated in A can be used as an evaluation adjunct in the following manner:

Minimise the error function, which can e.g. look like this:

$$E = {\sum }_{m = 1}^{{m}_{max}} {[ \vert U ( {p}_{m} ) - M ( {p}_{m} ) \vert * w ( {p}_{m} ) ]}^{2}$$

in which the error e (pₘ)=U(pₘ)−M(pₘ), i.e. the difference between the actual outcome and the target function.

Has the number of patients coming to and undergoing the examination been sufficient to permit the drawing any statistical conclusions about the system constants kᵢₓ(p)?

In which case, calculate how many more or fewer patients are required in each category in correcting deviations from previous rounds. Then add this number to subsequent rounds of summoned patients.

Repeat this until all the measurement values in the target function have been met or the aggregate weighted error is less than a limit value.

On the basis of these rules, steps 40, 42, 44, 46, 48 and 50 can then be repeated, again and again, as long as corrections are feasible in practice, i.e. as long as it is possible to book patients in vacant appointment slots. The system can also decide to add new appointment slots if this is necessary for meeting the target function.

In this manner, an adaptive process is achieved which steers operations towards the target.

Summary Description of the Algorithm's Use

1. Determine the target function's different parameters and the appointments at which the values for related variables are known.

2. Determine the relevant boundary conditions and the appointments at which the values for related variables are known.

3. Determine the error function E related to the target function and the associated weighting function w( ). Balance the weighting function's values, so deviations from desired values or ranges for the target function's different parameters are correctly counterbalanced.

4. Estimate the system constants Kᵢ(pₘ) for each step up to the start of treatment and within each step for each parameter in the target function. Using reverse arithmetic, this yields a first assumption about the number of patients, Nₛ₀, which must be summoned to screening.

5. Apply the boundary condition for known parameters to the patients constituting the pool of patients from which the selection is to be made. This results in a decimated number of patients from whom a selection will be summoned to an appointment.

6. Select the number of patients to be summoned to examination, corresponding to the number of appointment slots available for the period covered by the summons. The proportions must be right between each patient category that can be identified from the target function parameters available before the examination for which the selection is made. The number of patients is governed by the system constants for the examination, in question and by the magnitude of the accumulated error for the corresponding parameter. If a plurality of parameters in the target function are unable to meet their targets simultaneously, use the error function to assess the parameter to be assigned the highest priority.

7. Summon the patients who have been selected.

8. Check to ensure that a sufficiently number of patients has been examined and approved after the examinations. If this is the case, use the data for summoned and subsequently approved patients for adjusting system constants.

9. Repeat points 6 to 8 for each new period and examination to which patients are to be summoned.

One Example of a Study to which the Method and Algorithm Could be Applied

We shall now describe a multicentre study whose purpose is to demonstrate the hypotensive properties of drug X. It is to be performed on patients over 18, 50% of whom women. At the same time, we wish to avoid patients who are too old, as the risks are assumed to increase unacceptably. So we set an upper age limit of 75 years.

Patients with the highest possible blood pressure are desirable, as it is assumed that the effect will be most apparent in patients with hypertension. We want at least 25% of the patients have a systolic blood pressure greater than 140 mmHg.

In order to obtain statistically significant results proving the drug's efficacy, we will need 10,000 patients to start treatment.

The patients are randomised into two groups, patients in one group receiving the active drug X and patients in the other group receiving a placebo. Randomisation is also performed by central software.

Randomisation of patients into the respective group is performed on patients included at the start of treatment. Since patients randomly receive the placebo. the inclusion of patients with excessively high blood pressure may be inappropriate for ethical reasons. The limit for inclusion is set at a BPS of 180 mmHg.

All conditions apply to the study data as a whole. Distributions are allowed to vary from one centre to another.

We begin by defining the boundary conditions and the time at which data for the exclusion of inclusion of patients are to be available.

Boundary Conditions:

Boundary condition 1 (r₀₁): Age at least 18 years. Available before screening.

Boundary condition 2 (r₀₂): Age no more than 75 years. Available before screening.

Boundary condition 3 (r₁₁): Patients with a BPS over 180 mmHg to be excluded from this study. Available after screening (before the start of treatment).

We then define target function parameters and the respective time at which values are to be available for controlling them.

Target Function:

Parameter 1 (p₁): At least 10,000 patients must begin treatment. Available before screening, as the total number is in proportion to the number summoned to screening.

Parameter 2 (p₂): 50% of the patients must be women. Available before screening.

Parameter 3 (p₃): At least 25% of the patients must have a BPS greater than 140 mmHg. Available after screening (before the start of treatment).

Parameter 4 (p₄): The number of days the screening period is to last. 40 days or 8 weeks in our case. This is derived from the summons of 60,000 patients (cf. system constants below) to screenings at 50 contacted centres, each with a capacity of 30 patients a day.

Determine the error function E related to the target function and the associated weighting function w(pₘ).

Error function:

Call the error for our parameter pₘe(pₘ)=U(pₘ)−M (pₘ) where m=1,2,3,4

Select the error function:

$$E = {\sum }_{m = 1}^{4} {( \vert e ( {p}_{m} ) \vert * w ( {p}_{m} ) )}^{2}$$

Weighting function w(m):

w(1) is set at infinity for e(1)<0; 0.1 for e(1)>0

w(2) is set at 100(%)⁻¹ for e(2)

w(3) is set at infinity for e(3)<0; 0(%)^{-1 for} e(3)>0

w(4) is set at 0 for e(4)<0; 15 for e(4)>0

System constants K₁, K₂ are estimated below.

We initially approximate different patient categories, also referred to as subgroups, with the same values for K.

We estimate that 10% of the patients summoned to screening will be no-shows. This gives us k₁=0.9.

Epidemiological data show that about 5% of the populations suffers from hypertension. For this reason alone, we need to summon 5 times as many patients to achieve a target value of 25% for the parameter, thus k₂=0.2.

We estimate that 5% of the patients summoned to the start of treatment will be no-shows. Thus: k₃=0.95.

Since we do not know of any factors excluding patients at the start of treatment,

k₄=1.0.

K₁=k₁·k₂=0.9·0.2=0.18

K₂=k₃·k₄=0.95·1.0=0.95

With the end target of p₁=10,000 patients, the first estimate is obtained of the number of patients who need to be summoned. Calculate K=k₁·k₂·k₃·k₄=0.171. The number to be summoned to screening, Ns, then becomes 100,000/0.171=58,480, rounded off to 60,000. This figure is manually calculated the first time. The program calculates more accurate values as feedback is received from completed examinations.

The actual basic programming is now complete, and we can begin working with the patients.

The results of ads and Internet applications give us a sufficiently large pool of patients Pᵣ₀ in our database, e.g. the data storage unit 12.

Apply Boundary Conditions

One program run excludes patients outside the age limits defined in boundary conditions r₁ and r₂. We are initially unable to do anything about r₃, as blood pressures are not available until the first screening appointment.

Patients who fail to meet r₃ at the screening appointment are excluded.

Select Patients to be Summoned

We have decided that patients are to be sent summonses 2 weeks before the examination. In order to get screening started, the program randomly selects 1,500 patients a day for 2 weeks, with an even gender distribution, distributed among the participating centres. This is because we have adopted the same system constants for women as for the average.

To obtain more information, we then have to wait until a number of patients have had their screening appointments.

Adjust the System Constants

When two days have passed, 3,000 patients minus no-shows will have been screened. This is sufficient for checking to determine whether the system constants need adjustment. We find e.g. that more women than men show up for examination, but fewer women than men have blood pressures (BPS) exceeding 140 mmHg. The computer program will then calculate the new constants.

The above account has e.g. described the method according to the present invention with a multi-centre trial, but it should be noted that it can, in principle, be employed at a single centre.

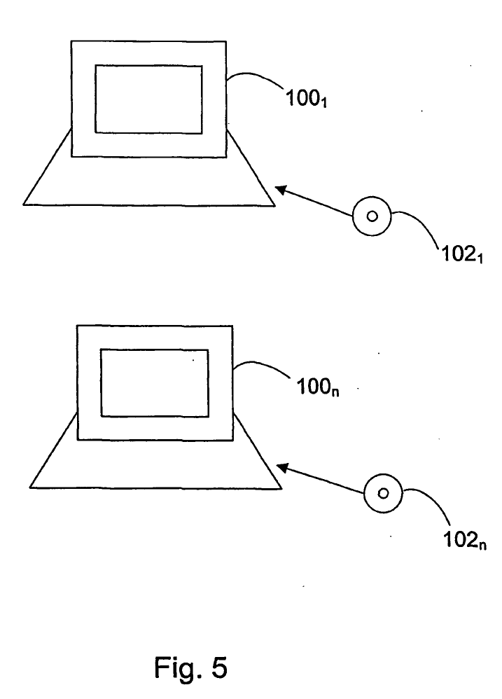

FIG. 5 is a schematic view of several computer program products according to the present invention. It shows n number of digital computers 100₁, . . . , 100ₙ. The various computer program products 102₁, . . . , 102ₙ can be downloaded into the internal memory of the various digital computers 100₁, . . . , 100ₙ. Each computer program product 102₁, . . . , 102ₙ comprises software components for performing some or all the steps according to FIG. 4 when the product/products 102₁, . . . , 102ₙ are run on the computer/computers 100₁, . . . , 100ₙ. The computer program products 102₁, . . . , 102ₙ can e.g. be in the form of diskettes, CD discs, RAM discs, magnetic tape, opto-magnetic discs or any other suitable product.

The invention is not restricted to the aforementioned embodiments. It will be apparent to anyone well-versed in the art that many different modifications are possible within the scope of the following claims.

1. A control system (10) intended for use in the performance of clinical trials, characterized in that the control system (10) comprises at least one data storage unit (12) for storing patient information and recorded or calculated data, one data acquisition means (14), connected to at least one of the said data storage units (12), that at least collects data excluding and/or including patients, a control means (16), connected to at least one of the said data storage units (12), that, on the basis of the collected data, books patient appointments or excludes patients through the use of at least one predefined boundary condition and/or predefined target function, M(p), in which p designates different parameters, and an appointment-booking means (18), connected to the control means (16), for booking patient appointment times, no recruitment of new patients to a clinical trial therefore being necessary when the control means (16) determines that the target function M(p) has been met or an error function E=f(M, U) is less than a predetermined threshold value, U(p) designating the actual outcome.

2. A control system (10) intended for use in performing clinical trials according to claim 1, characterized in that the error function is
$$E = {\sum }_{m = 1}^{{m}_{max}} {[ \vert U ( {p}_{m} ) - M ( {p}_{m} ) \vert * w ( {p}_{m} ) ]}^{n}$$
in which U(pₘ) is the actual outcome, n≧1 and w(pₘ) is a weighting function designating the degree of importance of meeting the target value for the parameter pₘ.

3. A control system (10) intended for use in the performance of clinical trials according to claim 1 or 2, characterized in that the initial part of each clinical trial is performed in three consecutive phases, a first phase in which the control means selects a subset, Pᵣ₁, of possible and/or desirable patients from a set Pᵣ₀ of interested participants stored in the data storage unit, a second phase in which a number of patients, Nₛ₀, from the set Pᵣ₁ of possible patients are summoned, in one or more steps, to an examination, whereupon the control means (16), on the basis of examinations results, excludes unsuitable patients, resulting in a set, Pₛ, of patients, and a third phase in which a number of patients, N_{b0}, from the set, Pₛ, are summoned to the start of treatment, whereupon the control means (16), on the basis of examination results, excludes unsuitable patients, resulting in a set P_{b}, of patients who receive treatment.

4. A control system (10) intended for use in the performance of clinical trials according to claim 3, characterized in that the number of patients, N.D, initially summoned is determined by the following relationship:
Nₛ₀=P₁/(K₁*K₂*K₃* . . . *Kₙ)
in which P₁ designates the number of patients who need to start treatment in the said clinical trial and Kᵢₓ, where 1≦i≦n, n being an integer, and 1≦x≦m, m being an integer with x designating different patient categories, designates an estimated system constant indicating the percentage of patients who were not excluded after each step, i, and 0≦Kᵢₓ≦1.

5. A control system (10) intended for use in performing clinical trials according to claim 4, characterized in that the control means corrects the system constants, Kᵢₓ, after a sufficient number of patients have undergone one or more of the said steps in claim 4.

6. A control system (10) intended for use in performing clinical trials according to any of claims 1-5, characterized in that the control means (16) adjusts the number and composition of patients summoned to examination until the clinical trial's treatment start is regarded as concluded.

7. A control system (10) intended for use in performing clinical trials according to any of the claims 1-6, characterized in that the said parameters can e.g. be the number of patients who begin treatment, gender distribution, age distribution and patient composition based on other patient values.

8. A control system (10) intended for use in performing clinical trials according to any of claims 1-7, characterized in that the said boundary conditions can be e.g. that the age of patients must lie within open or closed predefined intervals, that various patient values, such as laboratory results, must lie within open or closed predefined ranges, that certain diagnoses are met or not met etc.

9. A control system (10) intended for use in performing clinical trials according to any of claims 1-8, characterized in that at least one of the said data storage units (12) consists of a database.

10. A method intended for use in performing clinical trials, said method comprising the following steps:
Defining a target function M(p) encompassing a number of target values, p designating different parameters;
Estimating system constants, Kᵢₓ, in which 1≦i≦n, n being an integer, and 1≦x≦m, m being an integer and x designating different patient categories indicating the % of patients who, after each step, i, in the clinical trial were not excluded, and in which 0≦Kᵢₓ≦1;
Defining a data set for acquisition in each examination;
Defining a number of boundary conditions, rᵢⱼ, in which i designates the examination number, i, 1≦i≦n, n being an integer, j designating different boundary conditions for the examination, i, and 1≦j≦m, m being an integer;
Selecting a set, Pᵣ₁, of possible patients, from at least one of the data storage units holding patient data whose magnitude is selected on the basis of the number of patients who need to undergo treatment in the said clinical trial, and at least one exclusion criterion;
Selecting patients, with the aid of boundary conditions, rᵢⱼ, for the next examination number, i+1;
Including suitable patients, on the basis of examination results, resulting in a set, Pₛ, of patients;
Correcting the system constants, Kᵢₓ, on the basis of the actual outcome U(p);
Selecting patients from the set Pₛ to be summoned to the start of treatment, N_{b0};
Applying boundary conditions, rᵢⱼ, and, depending on measurement results from the examination at the start of treatment, excluding or including patients, resulting in a set, P_{b}, of patients to receive treatment.
Correcting, if necessary, the system constants, Kᵢₓ, on the basis of the actual outcome U(p);
Repeating the six preceding steps until the target function M(p) has been met or an error function E=f(M, U) is less than a predefined threshold value, U(p) designating the actual outcome.

11. A method intended for use in performing clinical trials according to claim 10, characterized in that the error function is
$$E = {\sum }_{m = 1}^{{m}_{max}} {[ \vert U ( {p}_{m} ) - M ( {p}_{m} ) \vert * w ( {p}_{m} ) ]}^{n}$$
where U(pₘ) is the actual outcome, M(Pₘ) is the target function, n>1 and w(pₘ) is a weighting function designating the degree of importance of reaching the parameter pₘ's target value.

12. A method intended for use in performing clinical trials according to either of claims 10 or 11, characterized in that an initially summoned number of patients, Nₛ₀, is determined by the following relationship:
Nₛ₀=P₁/(K₁*K₂*K₃* . . . *Kₙ).

13. A method intended for use in performing clinical trials according to any of claims 10-12, characterized in that an error function, E=f(M,U), is used for evaluating the priority of the parameters, pₘ, when a plurality of parameters in the target function, M(pₘ), are unable to reach their targets simultaneously.

14. A method intended for use in performing clinical trials according to any of claims 10-13, characterized in that the clinical trial can be devised as a multicentre trial.

15. At least one computer program product (102₁, . . . , 102ₙ), downloadable into the internal memory of at least one digital computer (100₁, . . . , 100ₙ), comprising software code for performing the steps according to claim 10 when at least one of the said products (102₁, . . . , 102ₙ) is run on at least one of the said computers (100₁, . . . , 100ₙ).
